(19) United States
(12) Patent Application Publication
(10) Pub. No.: US 2014/0305438 A1
(43) Pub. Date: Oct. 16, 2014

(54) PATIENT INTERFACE DEVICE WITH CUSHION SEALING ARRANGEMENT

(71) Applicant: KONINKLIJKE PHILIPS N.V., EINDHOVEN (NL)

(72) Inventor: Adam Michael Neff, Oakmont, PA (US)

(21) Appl. No.: 14/356,616

(22) PCT Filed: Oct. 31, 2012
(86) PCT No.: PCT/IB2012/056057

§ 371 Date: May 7, 2014

Related U.S. Application Data
(60) Provisional application No. 61/557,491, filed on Nov. 9, 2011.

(51) Int. Cl. A61M 16/06

(52) U.S. Cl. CPC A61M 16/0622; A61M 16/0638
USPC 128/206.24; 29/428

(57) ABSTRACT

A patient interface device (8) includes a frame (14) including a faceplate (18) having an outer surface (18A) and an inner surface (18B), the inner surface having an inward facing portion (74) which is oriented generally perpendicular to a patient's face when disposed on a patient's head. A cushion assembly (16) is coupled to the frame. The cushion assembly includes a support frame (44) including a coupling portion (48) having a bottom surface (48A) and an opposite top surface (48B) and a sealing cushion (46) coupled to the coupling portion of the support frame. The sealing cushion includes a first portion (62) extending from the bottom surface of the coupling portion and a second portion (64) extending from the top surface of the coupling portion, wherein an outward facing portion (72) of the second portion of the sealing cushion sealingly engages the inward facing portion of the frame.

### CROSS-REFERENCE TO RELATED APPLICATIONS

This patent application claims the priority benefit under 35 U.S.C. §119(e) of U.S. Provisional Application No. 61/557,491 filed on Nov. 9, 2011, the contents of which are herein incorporated by reference.

### BACKGROUND OF THE INVENTION

1. Field of the Invention

The present invention relates to respiratory therapy systems, such as non-invasive ventilation and pressure support systems, and in particular to a patient interface device for a respiratory therapy system that includes an improved sealing arrangement. The present invention also relates to a method of assembling a patient interface device.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is obstructive sleep apnea (OSA). Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient. It is known to maintain such devices on the face of a wearer by a headgear having one or more straps adapted to fit over/around the patient's head.

Adherence and compliance to therapy, such as CPAP or other pressure support therapies, is growing to be an industry-wide issue. Factors such as comfort and ease of using/changing components of a patient interface device can greatly affect a patient's adherence and compliance to therapy.

A certain amount of sealing force is required to attach a removable sealing/support cushion to a faceplate on a CPAP mask. The force is generally dependent on the pressure that the mask has to withstand before leaking. In current patient interface devices, the force is usually obtained through a compressive force by squeezing silicon between two ridged components. Another known approach utilizes a ridged groove in the faceplate into which a portion of the silicon cushion is pressed. The drawback to such methods is they require a lot of flat surface where the cushion and mask interface, thus making the mask wider/taller and covering a larger area of a patient's face.

### SUMMARY OF THE INVENTION

In one embodiment, a patient interface device is provided that includes a frame member including a faceplate portion having an outer surface and an inner surface opposite the outer surface. The inner surface has an inward facing portion adapted to be oriented generally perpendicular to a patient's face when disposed on a patient's head. The patient interface device also includes a cushion assembly coupled to the frame member. The cushion assembly includes a support frame including a coupling portion having a bottom surface and an opposite top surface, and a sealing cushion coupled to the coupling portion of the support frame, the sealing cushion having a first portion extending from the bottom surface of the coupling portion a second portion extending from the top surface of the coupling portion. An outward facing portion of the second portion of the sealing cushion sealingly engages the inward facing portion of the frame member.

In another embodiment, a method of assembling a patient interface device is provided that includes holding a frame member including a faceplate portion having an outer surface and an inner surface opposite the outer surface, the inner surface having an inward facing portion adapted to be oriented generally perpendicular to a patient's face when disposed on a patient's head. The method further includes holding a cushion assembly including a support frame and a sealing cushion coupled to a coupling portion of the support frame. The sealing cushion having a first portion extending from a bottom surface of the support frame and a second portion extending from a top surface of the support frame. The method also including coupling the cushion assembly to the frame member by sealingly engaging an outward facing portion of the second portion of the sealing cushion with the inward facing portion of the frame member.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic diagram of a system adapted to provide a regimen of respiratory therapy to a patient according to one exemplary embodiment of the present invention;

FIG. 2 is a front isometric view and FIG. 3 is a rear elevational view of a frame member forming a part of a patient interface device of the system of FIG. 1;

FIG. 4 is an isometric view of a cushion assembly forming a part of a patient interface device of the system of FIG. 1;

FIG. 5 is an isometric view and FIG. 6 is a front elevational view of a support frame forming a part of the cushion assembly of FIG. 4;

FIG. 7 is a vertical cross sectional view of the patient interface device of FIG. 1 taken along line 7-7 of FIG. 1;

FIG. 8 is a detail view of the portion of FIG. 7 indicated by line 8-8 in FIG. 7;

FIG. 9 is a detail view of the portion of FIG. 7 indicated by line 9-9 in FIG. 7;

FIG. 10 is a horizontal cross sectional view of the patient interface device of FIG. 1 taken along line 10-10 of FIGS. 1; and

FIG. 11 is an isometric view showing the manner in which the cushion assembly of FIG. 4 is attached to the frame member of FIGS. 2 and 3.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

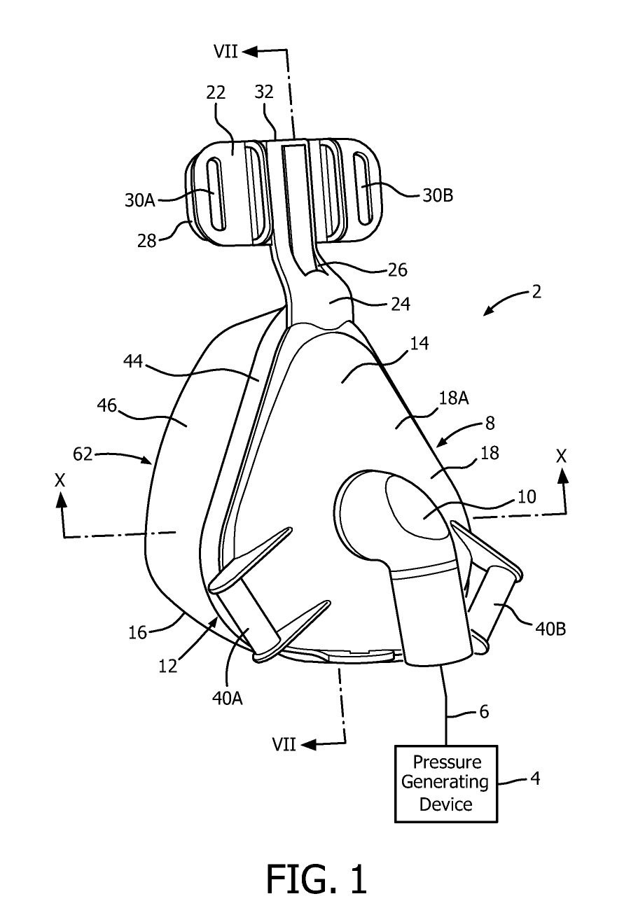

A system 2 adapted to provide a regimen of respiratory therapy to a patient according to one exemplary embodiment is generally shown in FIG. 1. System 2 includes a pressure generating device 4 (shown schematically), a patient circuit 6 (also shown schematically), and a patient interface device 8 having a fluid coupling conduit 10. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to patient interface device 8 through fluid coupling conduit 10, which in the illustrated embodiment is an elbow connector. Delivery conduit 6 and patient interface device 8 are often collectively referred to as a patient circuit.

In the exemplary embodiment, patient interface 8 includes a patient sealing assembly 12, which in the illustrated embodiment is a nasal/oral mask. However, other types of patient sealing assemblies, such as, without limitation, a nasal mask or a full face mask, which facilitates the delivery of the flow of breathing gas to the airway of a patient, may be substituted for patient sealing assembly 12 while remaining within the scope of the present invention. Patient sealing assembly 12 includes a frame member 14 having a cushion assembly 16 coupled thereto, each of which is described in greater detail below.

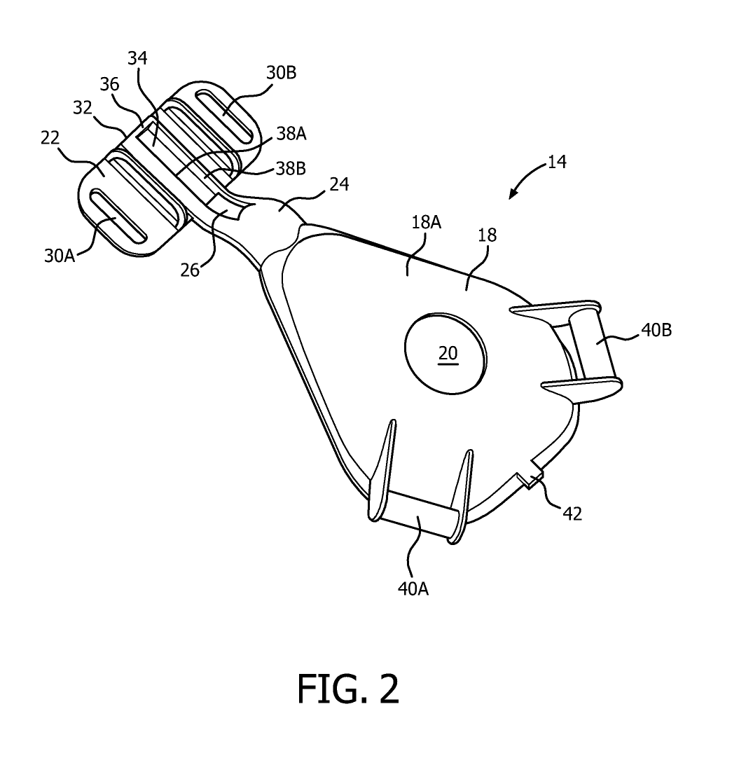

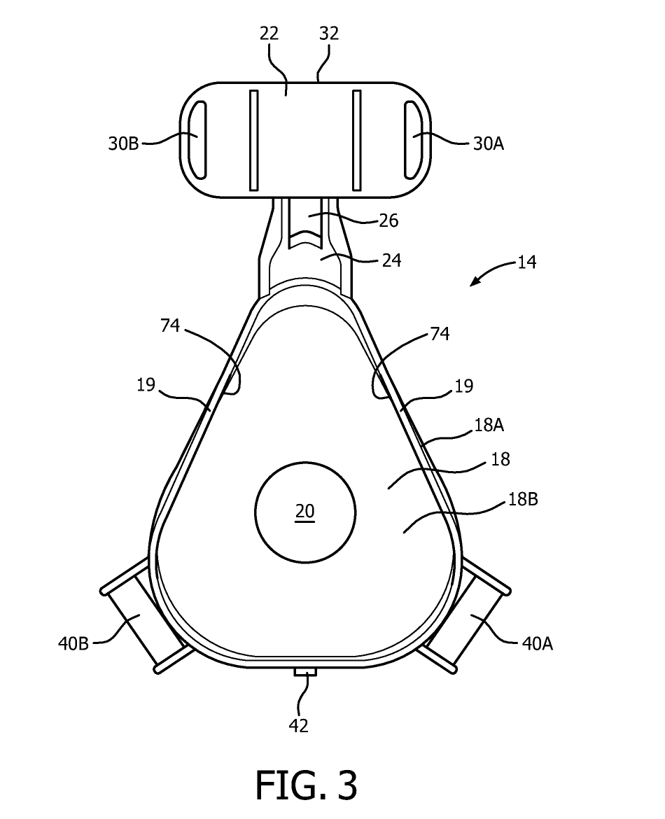

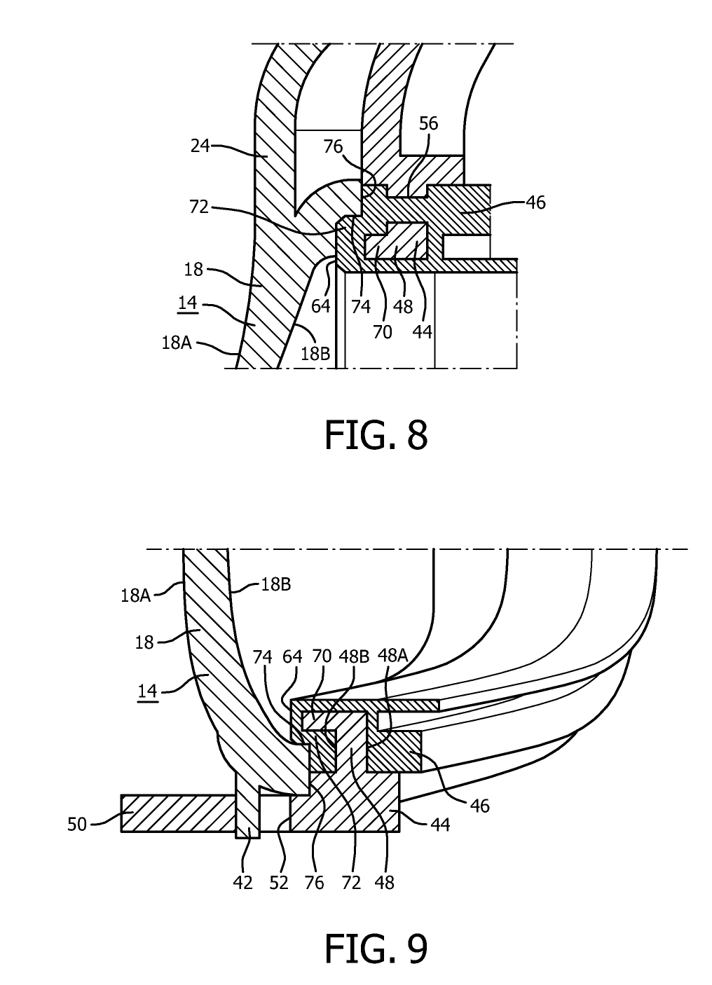

FIG. 2 is a front isometric view and FIG. 3 is a rear elevational view of frame member 14 according to the exemplary embodiment. In the illustrated embodiment, frame member 14 is made of a rigid or semi-rigid material, such as, without limitation, an injection molded thermoplastic, polycarbonate, or silicone, and includes a faceplate portion 18 having an outer surface 18A, an inner (patient facing) surface 18B opposite outer surface 18A, and an opening 20 formed therein and passing therethrough. Faceplate portion 18 terminates at a rear face 19 (FIGS. 3, 8 and 9) which spans between outer and inner surfaces 18A, 18B. As seen in FIG. 1, fluid coupling conduit 10 is coupled to faceplate portion 18 through via opening 20, which configuration allows the flow of breathing gas from pressure generating device 4 to be communicated to an interior space (not numbered) defined by inner surface 18B of faceplate portion 18 and cushion assembly 16, and then to the airway of a patient.

Frame member 14 also includes a forehead support member 22 that is coupled to faceplate portion 18 by a connecting member 24. An orifice 26 extends through connecting member 24, the function of which is described elsewhere herein. As seen in FIG. 1, a forehead cushion 28 is coupled to the rear of forehead support member 22. In the exemplary embodiment, forehead cushion 28 is made of a unitary piece of soft, flexible, cushiony, elastomeric material, such as, without limitation, silicone, an appropriately soft thermoplastic elastomer, a closed cell foam, or any combination of such materials. In addition, forehead support member 22 includes looped connectors 30A, 30B to which the upper straps of a headgear component (not shown) may be attached. Furthermore, the central portion 32 of forehead support member 22 includes a receiving channel 34 adjacent to orifice 26 defined by a top wall 36 and side walls 38A, 38B.

Connecting elements 40A, 40B are attached to the bottom of faceplate portion 18, and are structured to receive and hold the lower straps of a headgear component (not shown). In addition, a peg member 42 (FIG. 2) extends outwardly from the bottom of faceplate portion 18. The function of peg member 42 is described below.

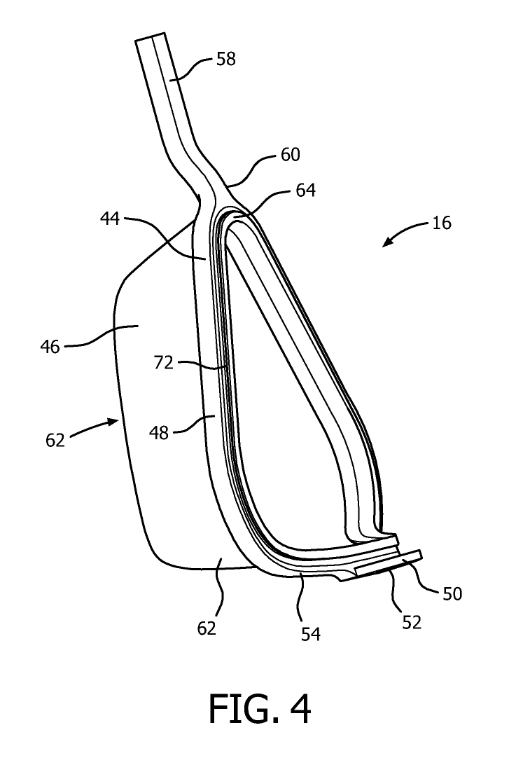

FIG. 4 is an isometric view of cushion assembly 16. Cushion assembly 16 includes a support frame 44 and a sealing cushion 46 coupled to support frame 44. In the illustrated embodiment, support frame 44 is made of a rigid or semi-rigid material, such as, without limitation, an injection molded thermoplastic or silicone, and sealing cushion 46 is defined from a unitary piece of soft, flexible, cushiony, elastomeric material, such as, without limitation, silicone, an appropriately soft thermoplastic elastomer, a closed cell foam, or any combination of such materials.

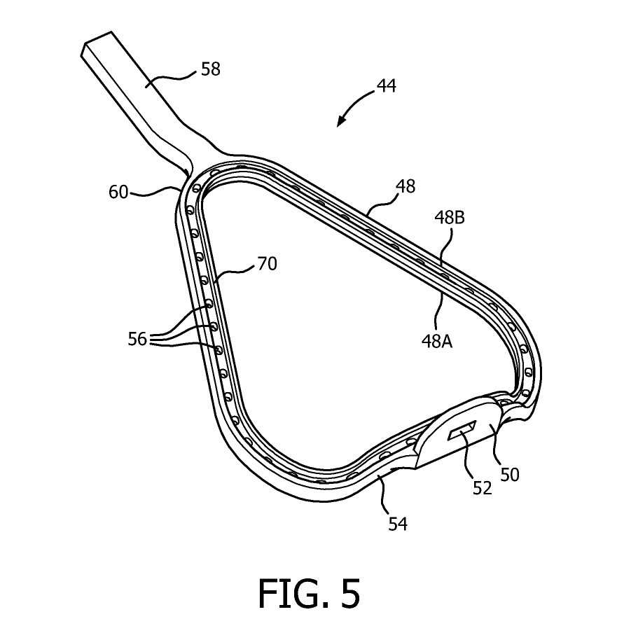

FIG. 5 is an isometric view and FIG. 6 is a front elevational view of support frame 44 according to the exemplary embodiment. Support frame 44 includes a generally triangular ring portion 48. A tab member 50 having a hole 52 provided therein is provided on the bottom end 54 of ring portion 48. Ring portion 48 also includes a plurality of holes 56 extending therethrough, the function of which is described below. A lever arm 58 extends from the apex 60 of ring portion 48. As seen in FIG. 4, sealing cushion 46 is coupled to ring portion 48. In the illustrated embodiment, sealing cushion 46 is overmolded onto ring portion 48 (through holes 56) such that a first portion 62 of sealing cushion 46, adapted to engage the face of the patient, extends from a bottom surface 48A of ring portion 48 and a second portion 64 of sealing cushion 46 extends from an opposite top surface 48B of ring portion 48. It is to be understood that the present invention contemplates that first portion 62 of sealing cushion 46 may be of any suitable structure for sealingly engaging the face of a patient.

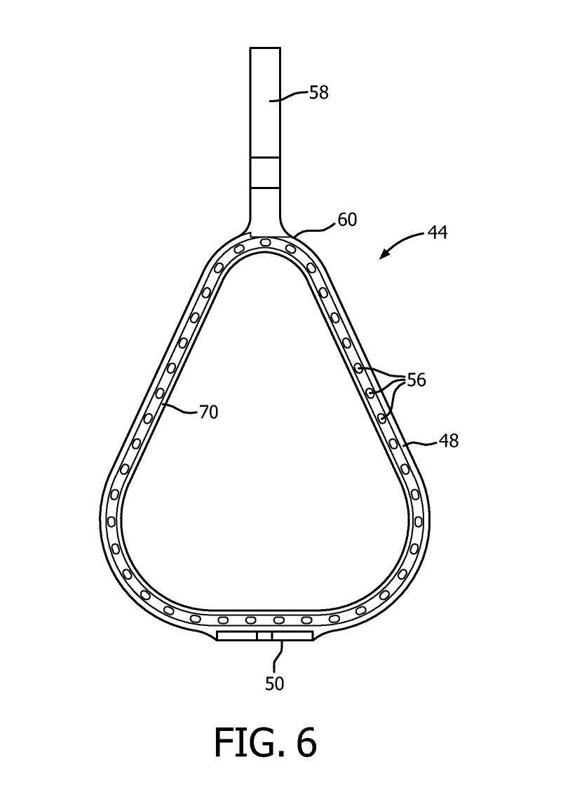

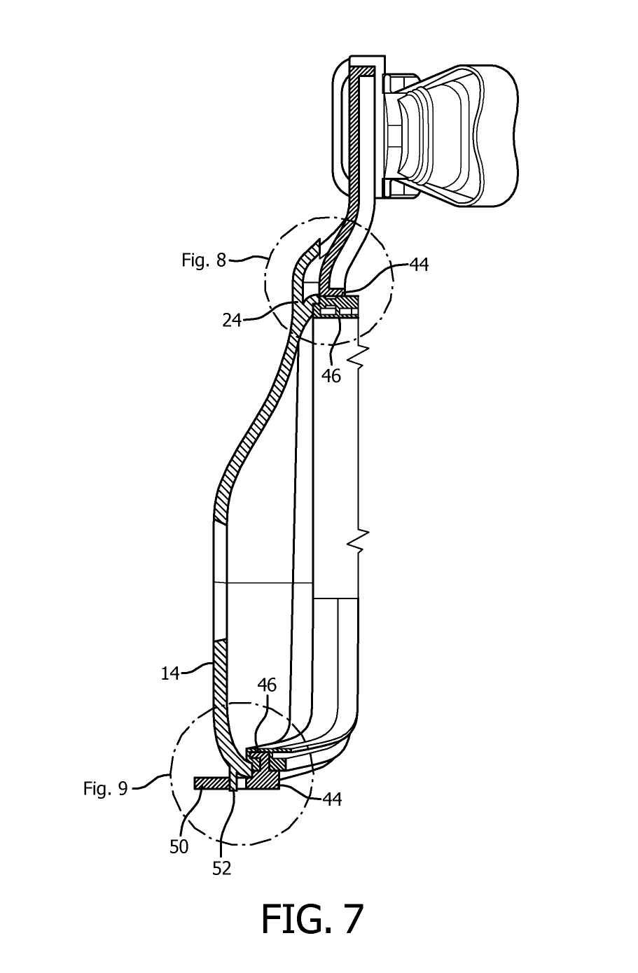

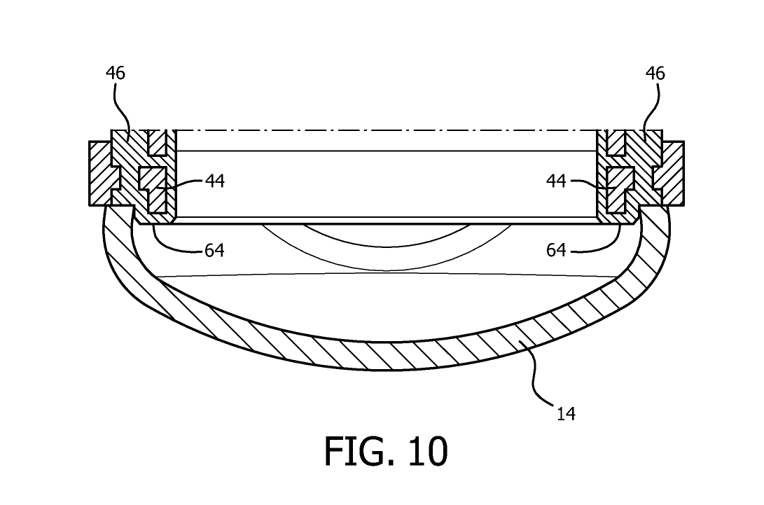

As shown in FIGS. 5 and 6 and the cross-sectional views of FIGS. 7-10, support frame 44 includes a ridged ring portion 70 extending from top surface 48B. When cushion assembly 16 is secured to frame member 14, as discussed below, ridged ring 70 acts to press an outward facing (with respect to ring portion 48 of support member 44) portion 72 of second portion 64 of sealing cushion 46 against an inward facing portion 74 of inner surface 18B of faceplate portion 18 of frame member 14. As shown in FIGS. 3, 8 and 9, inward facing portion 74 is disposed adjacent, and perpendicular to, rear face 19 of frame member 14.

The interaction of outward facing portion 72 of cushion 46 and inward facing portion 74 of inner surface 18B of frame member 14 forms an air-tight seal between cushion assembly 16 and frame member 14 (through compression of outward facing portion 72 of sealing cushion 46 between ridged ring portion 70 of support frame and 44 and inward facing portion 74 of inner surface 18B of faceplate portion 18). It is to be appreciated that outward facing portion 72 of cushion 46 and inward facing portion 74 of frame member 14 are oriented generally perpendicular to rear face 19 of frame member 14 (which is adapted to be oriented generally parallel to a patient's face when patient interface device 8 is positioned on the head of a patient). Accordingly, the sealing surface between frame member 14 and cushion assembly 16 is disposed generally perpendicular to the patient's face, hence minimizing the overall footprint of patient interface device 8 on the face of a patient.

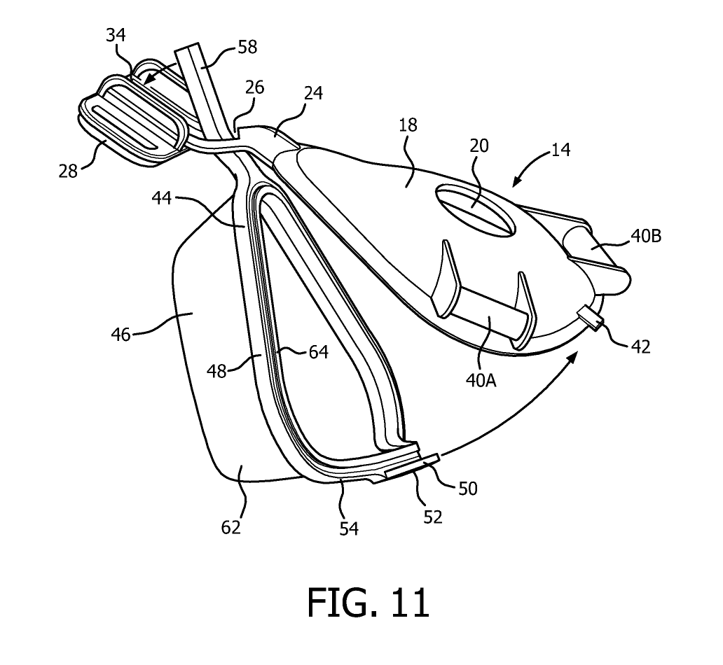

Cushion assembly 16 is removeably attached to frame member 14 in the following manner First, lever arm 58 of support frame 44 is inserted through orifice 26 of connecting member 24 of frame member 14. When this is done, the bottom of lever arm 58 will engage the front of receiving channel 34, which acts a pivot point for support frame 44. A force is then applied to lever arm 58 to cause cushion assembly 16 to rotate about the pivot point as shown by the arrows in FIG. 11. Sufficient force is applied to cause cushion assembly 16 to rotate to an extent that lever arm 58 is received in receiving channel 34 and engages the bottom surface thereof (FIG. 1). In addition, when this happens, peg member 42 is received in hole 52 of tab member 50 to secure cushion assembly 16 in place against frame member 14. When so secured, outward facing portion 72 of second portion 64 of sealing cushion 46 engages the inward facing portion 74 of faceplate portion 18 and is compressed between ridged ring portion 70 of ring portion 48 and inward facing portion 74 of faceplate portion 18 to create an airtight seal.

The insertion of peg member 42 into hole 52 produces a single audible click that indicates to the patient that the cushion assembly 16 has been properly attached to frame member 14. Receiving channel 34 may be sized to provide a friction fit between it and lever arm 58 to help secure cushion assembly 16 to frame member 14. Also, a releasable attachment mechanism, such as a detent or the like, may be provided on lever arm 58 or in receiving channel 34 to temporarily secure the two parts to one another. Cushion assembly 16 may be removed from frame member 14 by disengaging peg member 42 from hole 52 and rotating cushion assembly 16 in the opposite direction. While peg member 42 and hole 52 are shown as the exemplary locking mechanism, it will be appreciated that other suitable locking mechanisms, such as conventional snaps or cooperating magnets, may also be used.

In alternative embodiments, the surface that lever arm 58 engages does not need to be on the forehead support member 22. Instead, it may be on other portions of the frame member 14, such as, without limitation, the connecting member 24 or the faceplate portion 18. In such embodiments, the receiving channel 34 may be provided on that engagement surface.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A patient interface device comprising:
a frame member including a faceplate portion having an outer surface and an inner surface opposite the outer surface, the inner surface having an inward facing portion adapted to be oriented generally perpendicular to a patient's face when disposed on a patient's head; and
a cushion assembly coupled to the frame member, the cushion assembly comprising:
a support frame including a coupling portion having a bottom surface and an opposite top surface, and
a sealing cushion coupled to the coupling portion of the support frame, the sealing cushion having a first portion extending from the bottom surface of the coupling portion and a second portion extending from the top surface of the coupling portion, wherein an outward facing portion of the second portion of the sealing cushion sealingly engages the inward facing portion of the frame member,
wherein the frame member includes an orifice formed therein,
wherein the support frame includes a lever arm extending from the coupling portion, the lever arm being received through the orifice and engaging an engagement surface forming part of a front portion of the frame member,
wherein at least a first part of the coupling portion opposite the lever arm is releasably attached to a first part of the faceplate portion, and
wherein the first part of the coupling portion includes a first component of a releasable attachment mechanism and the first part of the faceplate portion includes a second component of the releasable attachment mechanism structured to be releasably attached to the first component.

2. The patient interface device of claim 1, wherein the support frame includes a ridged ring portion extending from the top surface of the coupling portion, the ridged ring portion being disposed to press the outward facing portion of the sealing cushion outward against the inward facing portion of the frame member.

3. The patient interface device of claim 2, wherein the outward facing portion of the second portion is compressed between the ridged ring portion of the coupling portion and the inward facing portion of the frame member.

4. The patient interface device of claim 1 wherein the cushion assembly is coupled to the coupling portion via over-molding.

5. The patient interface device of claim 1, wherein the coupling portion is of a generally annular shape.

6. The patient interface device of claim 5, wherein the frame member includes a forehead support member and a connecting member a extending between the faceplate portion and the forehead support member, wherein the orifice is formed in the connecting member; and wherein the engagement surface forms a part of a front portion of the forehead support member.

7. The patient interface device of claim 6, wherein the lever arm engages and is rotatable about a part of the front portion of the forehead support member.

8. The patient interface device of claim 7, wherein the front portion of the forehead support member comprises a receiving channel provided on the forehead support member, and wherein the lever arm engages and is rotatable about a front of the receiving channel.

9. The patient interface device of claim 8, wherein the receiving channel is defined by a top wall and first and second side walls provided on the forehead support member.

10-11. (canceled)

12. The patient interface device of claim 1, wherein the first component comprises a member having a hole provided therein, and the second component comprises a peg member structured to be received in the hole.

13. A method of assembling a patient interface device comprising:
holding a frame member including a faceplate portion having an outer surface and an inner surface opposite the outer surface, the inner surface having an inward facing portion adapted to be oriented generally perpendicular to a patient's face when disposed on a patient's head, the frame member also including an orifice formed therein;
holding a cushion assembly including a support frame and a sealing cushion coupled to a coupling portion of the support frame, the sealing cushion having a first portion extending from a bottom surface of the support frame and a second portion extending from a top surface of the support frame, the support frame having a lever arm extending from the coupling portion; and
coupling the cushion assembly to the frame member by
inserting the lever arm through the orifice,
rotating the cushion assembly relative to the frame member and causing the lever arm to engage an engagement surface forming a part of a front surface of the frame member, and
sealingly engaging an outward facing portion of the second portion of the sealing cushion with the inward facing portion of the frame member.

14. The method of claim 13 wherein the support frame includes a ridged ring portion extending from the top surface of the coupling portion, and wherein coupling the cushion assembly to the frame member comprises compressing the outward facing portion of the second portion of the sealing cushion between the ridged ring portion of the coupling portion and the inward facing portion of the frame member.

15. The method of claim 13, further comprising releasably attaching a first part of the coupling portion of the support frame opposite the lever arm to a first part of the faceplate portion generally opposite the orifice.

16. (canceled)

17. The method of claim 13, wherein the frame member includes a forehead support member and a connecting member extending between the faceplate portion and the forehead support member, wherein the orifice is provided in the connecting member, and wherein the engagement surface comprises a front portion of the forehead support member.

18. The method of claim 17, wherein the front portion of the forehead support member comprises a receiving channel provided on the forehead support member, and wherein rotating the cushion assembly causes the lever arm to be received within the receiving channel.
